(19) United States
(12) Patent Application Publication
(10) Pub. No.: US 2025/0134138 A1
(43) Pub. Date: May 1, 2025

(54) SILAGE INOCULANTS FOR INHIBITION OF ACETOBACTER

(71) Applicant: PIONEER HI-BRED INTERNATIONAL, INC., JOHNSTON, IA (US)

(72) Inventors: SCOTT MICHAEL DENNIS, URBANDALE, IA (US); JOHN C IIAMS, AMES, IA (US); WILLIAM C MAHANNA, JOHNSTON, IA (US); BRENDA SMILEY, GRANGER, IA (US)

(73) Assignee: PIONEER HI-BRED INTERNATIONAL, INC., JOHNSTON, IA (US)

(21) Appl. No.: 18/686,677

(22) PCT Filed: Aug. 29, 2022
(86) PCT No.: PCT/US2022/075563

§ 371 Date: Feb. 26, 2024

Related U.S. Application Data
(60) Provisional application No. 63/238,388, filed on Aug. 30, 2021.

(51) Int. Cl. A23K 30/18; A23K 10/12; C12N 1/18; C12N 1/20; C12R 1/225; C12R 1/24; C12R 1/25; C12R 1/865

(52) U.S. Cl. CPC A23K 30/18; A23K 10/12; C12N 1/185; C12N 1/205; A23V 2400/11; A23V 2400/121; A23V 2400/169; C12R 2001/225; C12R 2001/24; C12R 2001/25; C12R 2001/865

(57) ABSTRACT

A method for treating silage to enhance the aerobic stability by increasing the fermentation and stabilization of silage is disclosed. The method comprises treating silage or feed with a composition comprising one or more of a Lactobacillus brevis (Levilactobacillus brevis) strain LB7148, deposited as Patent Deposit No. NRRL B-67991 and Lactobacillus buchneri (Lentilactobacillus buchneri) strain LN7149, deposited as Patent Deposit No. NRRL B-67992, including mixtures or mutant(s) thereof which retain their silage preservative activity and/or or the anti-Acetobacter spp. components produced by LB7148 and LN7149, respectively. The strains of Lactobacillus buchneri (Lentilactobacillus buchneri) and Lactobacillus brevis (Levilactobacillus brevis) disclosed herein have been purified, isolated, and when applied to pre-ensiled plant material found to inhibit the growth of Acetobacter spp. and to improve aerobic stability of silage.

### FIELD OF THE DISCLOSURE

The present disclosure relates to compositions and methods of treating animal feed and preserving silage to enhance aerobic stability.

### BACKGROUND OF THE DISCLOSURE

The ensiling process is a method of moist forage preservation and is used worldwide. Silage accounts for more than 200 million tons of dry matter stored annually in Western Europe and the United States alone. The process involves natural fermentation, where lactic acid bacteria ferment water soluble carbohydrates to form organic acids under anaerobic conditions. This causes a decrease in pH which then inhibits detrimental microbes so that the moist forage is preserved.

Aerobic instability is the primary problem in silage production. Traditionally, the recommendation has been to allow silage to ferment for at least thirty (30) days before feeding to aid in increased silage digestibility. Even before silage storage units are opened for feedout, silage can be exposed to oxygen because of management problems (i.e., poor packing or sealing). Under these types of aerobic conditions, rapid growth of yeast and mold cause silage to heat and spoil, decreasing its nutritional value. Feeding an animal a crop that has not been properly fermented can lower dry matter intake (DMI), decrease milk production, and cause digestive upset. Allowing time for adequate fermentation creates a more palatable and digestible feed for optimum DMI and milk production.

Aerobic instability can be a problem even in inoculated silage that has undergone what would traditionally be considered a “good” fermentation: a rapid pH drop, and a low terminal pH. Silage inoculants containing a combination of both homofermentative lactic acid bacteria (to efficiently drop pH) and heterofermentative Lactobacillus buchneri (Lentilactobacillus buchneri) (to inhibit yeast) have proven to be an effective management tool for driving “front-end” fermentation and reducing “back-end” heating at feedout. The yeast organisms that contribute to instability in these conditions however may be those that are tolerant of acidic conditions and those that can metabolize the lactic acid produced by lactic acid bacteria during fermentation. Silage heating sometimes occurs even when yeast counts are low. While yeast are considered the main culprits of initiating heating when silage is exposed to air, it has recently been reported that Acetobacter spp. initiates heating as well (Mahanna and Dennis, Sep. 10, 2017, issue of Hoard's Dairyman, W. D. Hoard and Sons Company, Fort Atkinson, Wisconsin).

Acetobacter spp. are gram-negative aerobes that are very acid-tolerant, so low pH is not inhibitory to their survival. They are omnipresent in the environment, including in soil and water, and are airborne. Acetobacter spp. have the ability to preferentially convert ethanol to acetic acid in the presence of oxygen. They are also capable of converting lactic and acetic acids to carbon dioxide, water, and heat when ethanol levels are depleted. Acetobacter spp. and yeast often develop simultaneously when silage is exposed to air. Acetobacter spp. have been detected at relatively high levels (10E6-10E8 cfu/g) in problem silages where acetate was high, but heating was occurring. Some of these silages had been inoculated with L. buchneri-containing inoculants. In some cases, feed intake issues were also reported.

Production of silage inoculant strains and the ensiling process is complex and involves interactions of numerous chemical and microbiological processes. Different strains of even the same species do not have identical properties and vary in their fermentation and production characteristics. Further, different silages and different methods of ensiling present a variety of different needs. Therefore, a continuing need exists in the art for aerobic stability enhancing silage inoculants that are able to inhibit acetobacter, as well as yeast.

### SUMMARY OF THE DISCLOSURE

The present disclosure provides compositions and methods of using silage inoculants comprising silage quality preserving and Acetobacter spp. inhibiting heterofermentative lactic acid bacteria species, including mixtures or mutants thereof. The disclosed compositions can be used to inhibit undesirable effects of Acetobacter spp., improve the aerobic stability of ensiled forage, and increase the fermentation and stabilization of silage.

In an aspect, such compositions may include, but are not limited to, a first bacterial strain, wherein the first bacterial strain is any one of or a combination of Lactobacillus brevis (Levilactobacillus brevis) strain LB7148, deposited as Patent Deposit No. NRRL B-67991 and Lactobacillus buchneri (Lentilactobacillus buchneri) strain LN7149, deposited as Patent Deposit No. NRRL B-67992, a pre-ensiled plant material, and a suitable carrier. In particular examples of this aspect, the first bacterial strain (LB7148, LB7149 or a combination thereof) is prepared for packaging and storage by reversible inactivation, e.g., freeze drying or lyophilizing the first bacterial strain. Also provided is a package or container comprising reversibly inactivated first bacterial strain, which can further include a suitable carrier disclosed herein. The foregoing disclosed compositions may in some cases further comprise a second bacterial strain, wherein the second bacterial strain is selected from one or more of a Lactobacillus buchneri (Lentilactobacillus buchneri) strain, a Lactobacillus plantarum (Lactiplantibacillus plantarum) strain, a Lactobacillus alimentarius strain, a Lactobacillus crispatus strain, a Lactobacillus paralimentarius strain, a Lactobacillus brevis(Levilactobacillus brevis) strain, or an Enterococcus facium strain, including mixtures thereof. Thus, the second strain can include 1, 2, 3, 4, 5, 6, or 7 of the foregoing stains. In a further aspect, the compositions may further comprise a yeast strain, wherein the yeast strain is selected from one or more of Saccharomyces cerevisiae strain YE206, deposited as Patent Deposit No. NRRL Y-50734; Saccharomyces cerevisiae strain YE 1241, deposited as Patent Deposit No. NRRL Y-50735; or Saccharomyces cerevisiae strain YE1496, deposited as Patent Deposit No. NRRL Y-50736, and mixtures thereof. In an aspect, the second bacterial strain may comprise one or more of Lactobacillus plantarum (Lactiplantibacillus plantarum) strain LP287, Lactobacillus plantarum (Lactiplantibacillus plantarum) strain LP318, Lactobacillus plantarum (Lactiplantibacillus plantarum) strain LP319, Lactobacillus plantarum (Lactiplantibacillus plantarum) strain LP346, Lactobacillus plantarum (Lactiplantibacillus plantarum) strain LP347, Lactobacillus plantarum (Lactiplantibacillus plantarum) strain LP286, Lactobacillus buchneri (Lentilactobacillus buchneri) strain LN4017, Lactobacillus buchneri (Lentilactobacillus buchneri) strain LN4637, Lactobacillus plantarum (Lactiplantibacillus plantarum) strain LP329, Lactobacillus plantarum (Lactiplantibacillus plantarum) strain LP7109, Lactobacillus buchneri (Lentilactobacillus buchneri) strain LN7125, Lactobacillus brevis (Levilactobacillus brevis) strain LB7123, Lactobacillus brevis (Levilactobacillus brevis) strain LB5328, or Lactobacillus buchneri (Lentilactobacillus buchneri) strain LN5689, including mixtures thereof. Thus, the second strain can include 2, 3, 4, 5, 6, or 7 of the foregoing stains. Combinations of the first and second bacterial strain can be reversibly inactivated, and/or packaged or stored in container with a suitable carrier as described above for the first bacterial strain.

In another aspect, provided are compositions that comprise the first bacterial strain (with or without the second bacterial strain described above) and pre-ensiled plant material, wherein the composition comprises from about 10¹ to about 10¹⁰ viable organisms of each strain (or both strains) in the first bacterial strain per gram of the pre-ensiled plant material. For example, these compositions may comprise from about 10³ to about 10⁶ viable organisms of each strain (or both strains) in the first bacterial strain per gram of the pre-ensiled plant material; or such compositions may comprise about 10¹, 10², 10³, 10⁴, 10⁵, 10⁶, 10⁷, 10⁸, 10⁹, or 10¹⁰ viable organisms of each strain (or both strains) in the first bacterial strain per gram of the pre-ensiled plant material. In an aspect, the pre-ensiled plant material is selected from the group consisting of grasses, maize, alfalfa, wheat, legumes, sorghum, sunflower, barley, grains, and mixtures thereof. In an aspect, the carrier useful in the compositions of the present disclosure may be a liquid or a solid, such as, but not limited to, calcium carbonate, starch, maltodextrin, and cellulose.

In an aspect, the present disclosure provides methods for treating a pre-ensiled plant material, the methods comprising adding to the pre-ensiled plant material: a first bacterial strain, wherein the first bacterial strain is any one of or a combination of Lactobacillus brevis (Levilactobacillus brevis) strain LB7148, deposited as Patent Deposit No. NRRL B-67991 and Lactobacillus buchneri (Lentilactobacillus buchneri) strain LN7149, deposited as Patent Deposit No. NRRL B-67992, and a suitable carrier. In an aspect, the methods of treating a pre-ensiled plant material may further comprise adding a second bacterial strain to the pre-ensiled plant material, wherein the second bacterial strain is selected from one or more of a Lactobacillus buchneri (Lentilactobacillus buchneri) strain, a Lactobacillus plantarum (Lactiplantibacillus plantarum) strain, a Lactobacillus alimentarius strain, a Lactobacillus crispatus strain, a Lactobacillus paralimentarius strain, a Lactobacillus brevis (Levilactobacillus brevis) strain, or an Enterococcus facium strain, including mixtures thereof. Thus, the second strain can include 2, 3, 4, 5, 6, or 7 of the foregoing stains. In a further aspect, the methods of treating a pre-ensiled plant material may further comprise adding to the pre-ensiled plant material a yeast strain, wherein the yeast strain is selected from one or more of Saccharomyces cerevisiae strain YE206, deposited as Patent Deposit No. NRRL Y-50734; Saccharomyces cerevisiae strain YE 1241, deposited as Patent Deposit No. NRRL Y-50735; or Saccharomyces cerevisiae strain YE1496, deposited as Patent Deposit No. NRRL Y-50736, and mixtures thereof. In an aspect, the second bacterial strain useful in the methods of treating a pre-ensiled plant material of the present disclosure may comprise one or more of Lactobacillus plantarum (Lactiplantibacillus plantarum) strain LP287, Lactobacillus plantarum (Lactiplantibacillus plantarum) strain LP318, Lactobacillus plantarum (Lactiplantibacillus plantarum) strain LP319, Lactobacillus plantarum (Lactiplantibacillus plantarum) strain LP346, Lactobacillus plantarum (Lactiplantibacillus plantarum) strain LP347, Lactobacillus plantarum (Lactiplantibacillus plantarum) strain LP286, Lactobacillus buchneri (Lentilactobacillus buchneri) strain LN4017, Lactobacillus buchneri (Lentilactobacillus buchneri) strain LN4637, Lactobacillus plantarum (Lactiplantibacillus plantarum) strain LP329, Lactobacillus plantarum (Lactiplantibacillus plantarum) strain LP7109, Lactobacillus buchneri (Lentilactobacillus buchneri) strain LN7125, Lactobacillus brevis (Levilactobacillus brevis) strain LB7123, Lactobacillus brevis (Levilactobacillus brevis) strain LB5328, or Lactobacillus buchneri (Lentilactobacillus buchneri) strain LN5689, and mixtures thereof. Thus, the second strain can include 2, 3, 4, 5, 6, or 7 of the foregoing stains. In an aspect, from about 10¹ to about 10¹⁰ viable organisms of each strain (or both) strains in the first bacterial strain per gram of the pre-ensiled plant material are useful in the methods of treating a pre-ensiled plant material of the present disclosure. In an aspect, from about 10³ to about 10⁶ viable organisms of each strain (or both strains) in the first bacterial strain per gram of the pre-ensiled plant material are useful in the methods of treating a pre-ensiled plant material of the present disclosure. Alternatively, such methods may comprise adding about 10¹, 10², 10³, 10⁴, 10⁵, 10⁶, 10⁷, 10⁸, 10⁹, or 10¹⁰ viable organisms of each strain (or both strains) in the first bacterial strain per gram of the pre-ensiled plant material. Pre-ensiled plant material useful in the methods of the present disclosure may be selected from the group consisting of grasses, maize, alfalfa, wheat, legumes, sorghum, sunflower, barley, grains, and mixtures thereof. In certain examples, the first bacterial strain (and optionally the second bacterial strain) are re-activated with water or an aqueous liquid prior to treating the pre-ensiled plant material. In certain examples, the carrier useful in the methods of treating a pre-ensiled plant material of the disclosure may be a liquid or a solid, such as, but not limited to, calcium carbonate, starch, maltodextrin, and cellulose. The disclosed methods of treatment can include spraying compositions comprising the first bacterial (and/or the second bacterial strain) onto the pre-ensiled plant material.

In an aspect, the present disclosure provides methods for improving meat and milk performance in an animal, the methods comprising feeding the animal silage, wherein the silage comprises a pre-ensiled plant material treated with an inoculant comprising a first bacterial strain, wherein the first bacterial strain is any one of or a combination of Lactobacillus brevis (Levilactobacillus brevis) strain LB7148, deposited as Patent Deposit No. NRRL B-67991 and Lactobacillus buchneri (Lentilactobacillus buchneri) strain LN7149, deposited as Patent Deposit No. NRRL B-67992, and a suitable carrier. In an aspect, the methods of improving meat and milk performance in an animal may further comprise adding to the inoculant a second bacterial strain, wherein the second bacterial strain is selected from one or more of a Lactobacillus buchneri (Lentilactobacillus buchneri) strain, a Lactobacillus plantarum (Lactiplantibacillus plantarum) strain, a Lactobacillus alimentarius strain, a Lactobacillus crispatus strain, a Lactobacillus paralimentarius strain, a Lactobacillus brevis (Levilactobacillus brevis) strain, or an Enterococcus facium strain, including mixtures thereof. Thus, the second strain can include 2, 3, 4, 5, 6, or 7 of the foregoing stains. In a further aspect, the methods of improving meat and milk performance in an animal may further comprise adding to the inoculant a yeast strain, wherein the yeast strain is selected from one or more of Saccharomyces cerevisiae strain YE206, deposited as Patent Deposit No. NRRL Y-50734; Saccharomyces cerevisiae strain YE 1241, deposited as Patent Deposit No. NRRL Y-50735; or Saccharomyces cerevisiae strain YE1496, deposited as Patent Deposit No. NRRL Y-50736, and mixtures thereof. In an aspect, the second bacterial strain added to the inoculant useful in the methods of improving meat and milk performance in an animal of the present disclosure may comprise one or more of Lactobacillus plantarum (Lactiplantibacillus plantarum) strain LP287, Lactobacillus plantarum (Lactiplantibacillus plantarum) strain LP318, Lactobacillus plantarum (Lactiplantibacillus plantarum) strain LP319, Lactobacillus plantarum (Lactiplantibacillus plantarum) strain LP346, Lactobacillus plantarum (Lactiplantibacillus plantarum) strain LP347, Lactobacillus plantarum strain (Lactiplantibacillus plantarum) LP286, Lactobacillus buchneri (Lentilactobacillus buchneri) strain LN4017, Lactobacillus buchneri (Lentilactobacillus buchneri) strain LN4637, Lactobacillus plantarum (Lactiplantibacillus plantarum) strain LP329, Lactobacillus plantarum (Lactiplantibacillus plantarum) strain LP7109, Lactobacillus buchneri (Lentilactobacillus buchneri) strain LN7125, Lactobacillus brevis (Levilactobacillus brevis) strain LB7123, Lactobacillus brevis (Levilactobacillus brevis) strain LB5328, or Lactobacillus buchneri (Lentilactobacillus buchneri) strain LN5689, including mixtures thereof. Thus, the second strain can include 2, 3, 4, 5, 6, or 7 of the foregoing stains. In an aspect, from about 10¹ to about 10¹⁰ viable organisms of each strain (or both strains) in the first bacterial strain per gram of the pre-ensiled plant material are useful in the methods of improving meat and milk performance in an animal of the present disclosure. In an aspect, from about 10³ to about 10⁶ viable organisms of each strain (or both) strains in the first bacterial strain per gram of the pre-ensiled plant material are useful in the methods of improving meat and milk performance in an animal of the present disclosure. Alternatively, this method can include feeding an animal silage that has been treated with about 10¹, 10², 10³, 10⁴, 10⁵, 10⁶, 10⁷, 10⁸, 10⁹, or 10¹⁰ viable organisms of each strain (or both strains) in the first bacterial strain per gram of pre-ensiled plant material to improve the animals meat and milk performance. In an aspect, the pre-ensiled plant material useful in the methods of improving meat and milk performance in an animal of the present disclosure is selected from the group consisting of grasses, maize, alfalfa, wheat, legumes, sorghum, sunflower, barley, grains, and mixtures thereof. In an aspect, the carrier useful in the methods of improving meat and milk performance in an animal of the disclosure may be a liquid or a solid, such as, but not limited to, calcium carbonate, starch, maltodextrin and cellulose.

### BRIEF DESCRIPTION OF THE DRAWINGS

FIG. 1 shows an ethidium bromide stained agarose gel containing total DNA profiles, digested with Eco RI and electrophoresed on 0.7% LE agarose in 1× TAE buffer, of each of the strains of the present disclosure namely, Lactobacillus brevis (Levilactobacillus brevis) NRRL B-67991 (LB7148) and Lactobacillus buchneri (Lentilactobacillus buchneri) NRRL B-67992 (LN7149), and compared to the total DNA profiles, digested with Eco RI and electrophoresed on 0.7% LE agarose in 1× TAE buffer of Lactobacillus buchneri (Lentilactobacillus buchneri) ATCC 202118 (LN3957), Lactobacillus brevis (Levilactobacillus brevis) NRRL B-30865 (LB1154), Lactobacillus buchneri (Lentilactobacillus buchneri) NRRL B-30866 (LN4888), Lactobacillus brevis (Levilactobacillus brevis) NRRL B-50731 (LB5328), Lactobacillus brevis (Levilactobacillus brevis) NRRL B-50732 (LB7123), Lactobacillus buchneri (Lentilactobacillus buchneri) NRRL B-50733 (LN7125), Lactobacillus buchneri (Lentilactobacillus buchneri) PTA-2493(LN1391), Lactobacillus buchneri (Lentilactobacillus buchneri) PTA-2494 (LN4637), Lactobacillus buchneri (Lentilactobacillus buchneri) PTA-2495 (LN4750), and Lactobacillus buchneri (Lentilactobacillus buchneri) PTA-6138 (LN4017). As shown in FIG. 1 each strain has a unique Eco RI total DNA profile. Each of these strains were shown to be unique and different strains one from another.

### DETAILED DESCRIPTION

The disclosures herein will be described more fully hereinafter, in which some, but not all possible aspects are shown. Indeed, disclosures may be embodied in many different forms and should not be construed as limited to the aspects set forth herein; rather, these aspects are provided so that this disclosure will satisfy applicable legal requirements.

Many modifications and other aspects disclosed herein will come to mind to one skilled in the art to which the disclosed compositions and methods pertain having the benefit of the teachings presented in the following descriptions. Therefore, it is to be understood that the disclosures are not to be limited to the specific aspects disclosed and that modifications and other aspects are intended to be included within the scope of the appended claims. Although specific terms are employed herein, they are used in a generic and descriptive sense only and not for purposes of limitation.

It is also to be understood that the terminology used herein is for the purpose of describing particular aspects only and is not intended to be limiting. As used in the specification and in the claims, the term “comprising” can include the aspect of “consisting of.” Unless defined otherwise, all technical and scientific terms used herein have the same meaning as commonly understood by one of ordinary skill in the art to which the disclosed compositions and methods belong. Many methods and materials similar, modified, or equivalent to those described herein can be used in the practice of the embodiments of the present disclosure without undue experimentation, the preferred materials and methods are described herein. In this specification and in the claims which follow, reference will be made to a number of terms which shall be defined herein.

Units, prefixes, and symbols may be denoted in their SI accepted form. Numeric ranges recited within the specification are inclusive of the numbers defining the range and include each integer within the defined range.

The article “a” and “an” are used herein to refer to one or more than one (i.e., to at least one) of the grammatical object of the article. By way of example, “an element” means one or more elements.

As used herein, “animal performance” means the yield of meat, milk, eggs, offspring, or work.

As used herein, “ensiling” or “ensiled” refers to an anaerobic fermentation process used to preserve forages, immature grain crops, and other biomass crops for feed and biofuels. In some embodiments, the process of ensiling comprises the steps of contacting forage with a microbial inoculant and storing the mixture in an anaerobic condition. In certain embodiments, the process of ensiling comprises the steps of storing forage in anaerobic condition in a manner so as to exclude air. Forage, having been inoculated with the microbial inoculant described elsewhere herein, is also packed and stored in a manner so as to exclude air. The moisture content of forage can be about 50% to about 80%, depending on the means of storage, the amount of compression, and the expected moisture loss during storage. Ensiling or storage can occur in silos, silage heaps, silage pits, silage bales, or any other method appropriate for ensiling and storing the chosen plant material for later use. Plant material with a microbial inoculant described elsewhere herein can be ensiled for any amount of time appropriate to produce silage at the desired maturity stage. In some embodiments, ensiling occurs for about 7, about 15, about 20, about 25, about 30, about 35, about 40, about 41, about 42, about 43, about 44, about 45, about 46, about 47, about 48, about 49, about 50, about 55, about 60, about 65, about 70 days, about 4 months, about 8 months, about 12 months, about 18 months, or about 24 months or any time period deemed suitable by the practitioner. The ensiling process can take place at any ambient temperature, for example at an ambient temperature from 0-45° C. The temperature of the plant material being ensiled may, however, increase above 45° C. Mature silage can be used for animal feed, frozen and stored for a later use, or added to a biogas generator for the production of biogas.

As used herein, “functional mutant” means a genetically modified referenced strain(s) whether such modification occurs naturally or is manmade, wherein the genetically modified referenced strain(s) retains at least 50% of the activity of the referenced strain(s). The genetic modification can be achieved through any means, such as but not limited to, chemical mutagens, ionizing radiation, transposon-based mutagenesis, or via conjugation, transduction, or transformation using the referenced strain(s) as either the recipient or donor of genetic material.

As used herein, the term “heterofermentative lactic acid bacteria species” shall be interpreted to include, but not limited to, leuconostocs, some lactobacilli, oenococci, and weissella species. Heterofermenters produce lactic acid, ethanol, acetic acid and carbon dioxide, with the proportions depending upon the substrates available.

As used herein, the term “homofermentative lactic acid bacteria species” shall be interpreted to include, but not limited to, some lactobacilli and most species of enterococci, lactococci, pediococci, streptococci, tetragenococci, and vagococci that ferment hexoses by the Embden-Meyerhof (E-M) pathway. Homofermentative denotes that lactic acid is the principal metabolite without the production of carbon dioxide. For each six-carbon sugar molecule, homofermentative lactic acid bacteria will produce two molecules of lactic acid.

As used herein, “isolated” means removed from a natural source including, but not limited to, uninoculated silage or other plant material.

As used herein, “microbial inoculant” or “inoculant” refers to a composition comprising at least one bacterial culture and a suitable carrier. A “combination microbial inoculant” or “combination inoculant” comprises at least 2, at least 3, at least 4, at least 5, at least 6, at least 7, or more bacterial cultures and a suitable carrier. Bacterial cultures comprise at least one bacterial strain and may comprise multiple bacterial strains, including for example, at least 2, at least 3, at least 4, at least 5, at least 6, at least 7, or more. Bacterial cultures useful in the methods and compositions disclosed herein include, but are not limited to, Lactobacillus brevis (Levilactobacillus brevis) strain LB7148, deposited as Patent Deposit No. NRRL B-67991 and Lactobacillus buchneri (Lentilactobacillus buchneri) strain LN7149, deposited as Patent Deposit No. NRRL B-67992 and combinations thereof. The Lactobacillus brevis (Levilactobacillus brevis) strain LB7148, deposited as Patent Deposit No. NRRL B-67991 and Lactobacillus buchneri (Lentilactobacillus buchneri) strain LN7149, deposited as Patent Deposit No. NRRL B-67992 useful in the methods and compositions of the present disclosure may be combined with other bacterial strains, including but not limited to, a Lactobacillus buchneri (Lentilactobacillus buchneri) strain, a Lactobacillus plantarum (Lactiplantibacillus plantarum) strain, a Lactobacillus alimentarius strain, a Lactobacillus crispatus strain, a Lactobacillus paralimentarius strain, a Lactobacillus brevis (Levilactobacillus brevis) strain, or an Enterococcus facium strain, and mixtures thereof. Specific bacterial strains that may be combined with the Lactobacillus brevis (Levilactobacillus brevis) strain LB7148, deposited as Patent Deposit No. NRRL B-67991 and Lactobacillus buchneri (Lentilactobacillus buchneri) strain LN7149, deposited as Patent Deposit No. NRRL B-67992 useful in the compositions and methods of the present disclosure include, but are not limited to, Lactobacillus plantarum (Lactiplantibacillus plantarum) strain LP287, Lactobacillus plantarum (Lactiplantibacillus plantarum) strain LP318, Lactobacillus plantarum (Lactiplantibacillus plantarum) strain LP3710 (Patent Deposit No. PTA-6136), Lactobacillus plantarum (Lactiplantibacillus plantarum) strain LP3779 (Patent Deposit No. PTA-6137), Lactobacillus plantarum (Lactiplantibacillus plantarum) strain LP319, Lactobacillus plantarum (Lactiplantibacillus plantarum) strain LP346, Lactobacillus plantarum (Lactiplantibacillus plantarum) strain LP347, Lactobacillus plantarum (Lactiplantibacillus plantarum) strain LP286, Lactobacillus buchneri (Lentilactobacillus buchneri) strain LN4017, Lactobacillus buchneri (Lentilactobacillus buchneri) strain LN4637, Lactobacillus plantarum (Lactiplantibacillus plantarum) strain LP329, Lactobacillus plantarum (Lactiplantibacillus plantarum) strain LP7109 (Patent Deposit No. PTA-6139), Lactobacillus buchneri (Lentilactobacillus buchneri) strain LN7125, Lactobacillus brevis (Levilactobacillus brevis) strain LB7123, Lactobacillus brevis (Levilactobacillus brevis) strain LB1154 (Patent Deposit No. NRRL B-30865), Lactobacillus brevis (Levilactobacillus brevis) strain LB5328, or Lactobacillus buchneri (Lentilactobacillus buchneri) strain LN5689, and mixtures thereof. In addition, yeast, with or without another bacterial strain, may also be combined with the Lactobacillus brevis (Levilactobacillus brevis) strain LB7148, deposited as Patent Deposit No. NRRL B-67991 and Lactobacillus buchneri (Lentilactobacillus buchneri) strain LN7149, deposited as Patent Deposit No. NRRL B-67992 useful in the methods and compositions disclosed herein including, but not limited to, Saccharomyces cerevisiae strain YE206, deposited as Patent Deposit No. NRRL Y-50734; Saccharomyces cerevisiae strain YE 1241, deposited as Patent Deposit No. NRRL Y-50735; or Saccharomyces cerevisiae strain YE1496, deposited as Patent Deposit No. NRRL Y-50736, and mixtures thereof.

As used herein, “pre-ensiled plant material” includes, but is not limited to, grasses, maize, alfalfa, wheat, ryegrass, cereals, oil seeds, sorghum, sunflower, barley, and mixtures thereof prior to fermentation. All of which can be treated successfully with the inoculants of the embodiments of the present disclosure. The inoculants of the embodiments of the present disclosure are also useful in treating high moisture corn (HMC).

As used herein, “oilseeds” includes, but is not limited to sunflower, canola, soy, and mixtures thereof.

As used herein, “purified” means that a bacterial species or strain is substantially separated from, and enriched relative to yeasts, molds, and/or other bacterial species or strains found in the source from which it was isolated.

The term “silage” as used herein is intended to include all types of fermented agricultural products, including but not limited to, grass silage, alfalfa silage, wheat silage, legume silage, sunflower silage, barley silage, whole plant corn silage (WPCS), sorghum silage, fermented grains, and grass mixtures, etc.

As used herein, the term “strain” or “strain(s)” shall be interpreted to include, but not limited to, any mutant or derivative of the various bacterial strains disclosed herein, for example, Lactobacillus brevis (Levilactobacillus brevis) strain LB7148, deposited as Patent Deposit No. NRRL B-67991 and Lactobacillus buchneri (Lentilactobacillus buchneri) strain LN7149, deposited as Patent Deposit No. NRRL B-67992 which retains the functional activity of improving aerobic stability of forage as described and defined by the compositions, methods, and examples disclosed herein.

Microorganisms have been isolated and purified which improve the aerobic stability of ensiled forage, increase the fermentation and stabilization of silage, and inhibit the growth of Acetobacter spp. Specific strain(s) of the species L. buchneri or L. brevis have been shown to enhance aerobic stability of silage by not only inhibiting the growth of Acetobacter spp., reducing lactic acid levels, and also by producing substances which are inhibitory to microorganisms that contribute to causing aerobic instability in silage.

The primary goal of ensiling forages is to conserve the maximum amount of original dry matter, nutrients, and energy in the crop for feeding at a later time. The process can be characterized by four general phases of silage fermentation.

Upon sealing an ensiling storage unit, the first phase of silage fermentation is aerobic, when oxygen is still present between plant particles and the pH is 6.0 to 6.5. These conditions allow for continued plant respiration, protease activity, and the activity of aerobic and facultative aerobic microorganisms.

The second phase of silage fermentation is fermentation, which lasts several days to several weeks after the silage becomes anaerobic. Lactic acid bacteria grow and become the primary microbial population thereby producing lactic and other organic acids and decreasing the pH to 3.8 to 5.0.

The third phase of silage fermentation is stability with few changes occurring in the characteristics of the forage so long as air is prevented from entering the ensiling storage unit.

The final phase of silage fermentation is feedout wherein the silage is ultimately unloaded from the ensiling storage unit and exposed to air. This results in reactivation of aerobic microorganisms, primarily yeast, molds, bacilli, and acetic acid bacteria which can cause spoilage.

Management techniques used to help prevent this condition, include but are not limited to, using care to pack the silage well during the ensiling process, including rapid filling, compaction, sealing, and face management and, also, using care in removing silage for feeding to minimize the aeration of the remaining silage.

The susceptibility of silage to aerobic deterioration is determined by physical, chemical, and microbiological factors. Management, including, but not limited to compaction and unloading rates largely effects the movement of oxygen into silage. During feedout, air can penetrate up to 1 meter behind the silage face so that exposure to oxygen is prolonged. Fermentation acids and pH inhibit the rate of microbial growth but spoilage rates are affected also by microbial numbers and the rate of aerobic microbial growth on available substrates.

Lactic acid bacteria (LAB) are present as part of the normal microflora on growing plants. LAB can be classified as one of two types depending upon their primary metabolic end products; homofermentative which produce only lactic acid from the metabolism of glucose and heterofermentative which produce lactic acid, ethanol, acetate, and CO₂. The occurrences of these types of LAB are quite variable in both type and number, from crop to crop, and from location to location.

Silage inoculants comprising principally homofermentative lactic acid bacteria have become the dominant additives in many parts of the world. Their function is to promote rapid and efficient utilization of a crop's water-soluble carbohydrates resulting in intensive production of lactic acid and a rapid decrease in pH, thus minimizing dry matter losses. However, homofermentative inoculants often have a negative effect on aerobic stability due to the conservation of readily available substrates used by spoilage organisms.

The use of heterofermentative lactic acid bacteria in an inoculant has gained recent favor. Increased levels of undissociated volatile fatty acids, such as acetate, may inhibit other microbes that initiate aerobic deterioration. Heterofermenters produce lactic acid, ethanol, acetic acid, and carbon dioxide. The proportions of lactic acid, ethanol, acetic acid, and carbon dioxide produced depends upon the substrates available. The acetate produced may inhibit deleterious organisms in the silage. Additionally, heterofermenters, such as Lactobacillus buchneri (Lentilactobacillus buchneri), are capable of metabolizing lactic acid to acetate and 1,2 propanediol under anaerobic conditions. With such mechanisms, one sixth of the carbon is lost to carbon dioxide during fermentation of glucose and one third of the lactic acid carbon is lost during anaerobic conversion to acetic acid. However a small loss of 1% or perhaps up to 2% of the dry matter is easily offset by much larger losses by that spoilage action of aerobic microorganisms. Different strains of even the same species do not have identical properties, vary in their ability to inhibit Acetobacter spp., and have differing fermentation characteristics. Some inoculants may also improve animal performance.

In embodiments of the present disclosure, the inhibition of Acetobacter spp. and other organisms responsible for spoilage is accomplished by treating the silage with organisms of the species L. buchneri or L. brevis, especially Lactobacillus brevis (Levilactobacillus brevis) strain LB7148, Patent Deposit No. NRRL B-67991 and/or Lactobacillus buchneri strain LN7149, Patent Deposit No. NRRL B-67992, and combinations thereof.

An embodiment of the disclosure is a microbial inoculant comprising Lactobacillus brevis (Levilactobacillus brevis) strain LB7148, deposited as Patent Deposit No. NRRL B-67991 and/or Lactobacillus buchneri (Lentilactobacillus buchneri) strain LN7149, deposited as Patent Deposit No. NRRL B-67992, and mixtures thereof that will inhibitAcetobacter spp., alter fermentation, and enhance stabilization of silage.

An embodiment of the disclosure is a biologically pure culture of Lactobacillus brevis (Levilactobacillus brevis) strain LB7148, deposited as Patent Deposit No. NRRL B-67991 and/or a biologically pure culture Lactobacillus buchneri (Lentilactobacillus buchneri) strain LN7149, deposited as Patent Deposit No. NRRL B-67992.

Further embodiments of the present disclosure include methods of treating animal feed or silage, comprising administering a silage inoculant comprising Lactobacillus brevis (Levilactobacillus brevis) strain LB7148, deposited as Patent Deposit No. NRRL B-67991 and/or Lactobacillus buchneri (Lentilactobacillus buchneri) strain LN7149, deposited as Patent Deposit No. NRRL B-67992 to the feed or silage at about 10¹ to about 10¹⁰ viable organisms to about 10³ to about 10⁶ viable organisms per gram of a pre-ensiled plant material, animal feed, or silage. Additionally, the present disclosure provides methods of improving animal performance, comprising feeding an animal an animal feed that has been inoculated with the silage inoculants as described herein.

Embodiments of the disclosure include methods for treating silage by inhibiting the growth thereon of Acetobacter spp. and of spoilage organisms selected from yeasts, molds and spore-forming bacteria, which comprises adding to a pre-ensiled plant material: a first bacterial strain, wherein the first bacterial strain is any one of or a combination of Lactobacillus brevis (Levilactobacillus brevis) strain LB7148, deposited as Patent Deposit No. NRRL B-67991 and Lactobacillus buchneri (Lentilactobacillus buchneri) strain LN7149, deposited as Patent Deposit No. NRRL B-67992, and a suitable carrier. The methods of treating a pre-ensiled plant material may further comprise adding a second bacterial strain to the pre-ensiled plant material, wherein the second bacterial strain is selected from one or more of a Lactobacillus buchneri (Lentilactobacillus buchneri) strain, a Lactobacillus plantarum (Lactiplantibacillus plantarum) strain, a Lactobacillus alimentarius strain, a Lactobacillus crispatus strain, a Lactobacillus paralimentarius strain, a Lactobacillus brevis (Levilactobacillus brevis) strain, or an Enterococcus facium strain, and mixtures thereof. The methods of treating a pre-ensiled plant material may also further comprise adding to the pre-ensiled plant material a yeast strain, wherein the yeast strain is selected from one or more of Saccharomyces cerevisiae strain YE206, deposited as Patent Deposit No. NRRL Y-50734; Saccharomyces cerevisiae strain YE 1241, deposited as Patent Deposit No. NRRL Y-50735; or Saccharomyces cerevisiae strain YE1496, deposited as Patent Deposit No. NRRL Y-50736, and mixtures thereof. The second bacterial strain useful in the methods of treating a pre-ensiled plant material of the present disclosure may comprise one or more of Lactobacillus plantarum (Lactiplantibacillus plantarum) strain LP678 (Patent Deposit No. PTA-6134), Lactobacillus plantarum (Lactiplantibacillus plantarum) strain LP287, Lactobacillus plantarum (Lactiplantibacillus plantarum) strain LP318, Lactobacillus plantarum (Lactiplantibacillus plantarum) strain LP319, Lactobacillus plantarum (Lactiplantibacillus plantarum) strain LP346, Lactobacillus plantarum (Lactiplantibacillus plantarum) strain LP347, Lactobacillus plantarum (Lactiplantibacillus plantarum) strain LP286, Lactobacillus buchneri (Lentilactobacillus buchneri) strain LN4017, Lactobacillus buchneri (Lentilactobacillus buchneri) strain LN4637, Lactobacillus plantarum (Lactiplantibacillus plantarum) strain LP329, Lactobacillus plantarum (Lactiplantibacillus plantarum) strain LP7109, Lactobacillus buchneri (Lentilactobacillus buchneri) strain LN7125, Lactobacillus buchneri (Lentilactobacillus buchneri) strain LN4888 (Patent Deposit No. NRRL B-30866), Enterococcus faecium strain EF301, Enterococcus faecium strain EF202, Lactobacillus brevis (Levilactobacillus brevis) strain LB7123, Lactobacillus brevis (Levilactobacillus brevis) strain LB5328, Lactobacillus reuteri (Limosilactobacillus reuteri) strain LR4933 (Patent Deposit No. NRRL B-30867), Lactobacillus crispatus LI2127 (Patent Deposit No. NRRL B-30868), Lactobacillus crispatus, strain LI2350 (Patent Deposit No. NRRL B-30869), Lactobacillus crispatus, strain LI2366 (Patent Deposit No. NRRL B-30870), Lactobacillus species unknown, strain UL3050 (Patent Deposit No. NRRL B-30871), or Lactobacillus buchneri (Lentilactobacillus buchneri) strain LN5689, and mixtures thereof. From about 10¹ to about 10¹⁰ viable organisms of the first bacterial strain per gram of the pre-ensiled plant material are useful in the methods of treating a pre-ensiled plant material of the present disclosure. Further, from about 10³ to about 10⁶ viable organisms of the first bacterial strain per gram of the pre-ensiled plant material are useful in the methods of treating a pre-ensiled plant material of the present disclosure. The pre-ensiled plant material useful in the methods of the present disclosure may be made from a variety of plant sources, including but not limited to grasses, maize, alfalfa, wheat, legumes, sorghum, sunflower, barley, grains, and mixtures thereof. The inoculants of the embodiments of the present disclosure may also be added to the silage upon storage. The silage may be ensiled in a variety of ways, including in the form of a silage bale, a silage bag, a silage bunker, a silage pit, a stave silo, or a silage pile. The methods of treating silage using the compositions of the embodiments include adding to the silage an Acetobacter spp. inhibiting amount of Lactobacillus brevis (Levilactobacillus brevis) strain LB7148, deposited as Patent Deposit No. NRRL B-67991 and/or Lactobacillus buchneri (Lentilactobacillus buchneri) strain LN7149, deposited as Patent Deposit No. NRRL B-67992, and a suitable carrier. The carrier useful in the methods of treating a pre-ensiled plant material of the disclosure may be a liquid or a solid, such as, but not limited to, calcium carbonate, starch, maltodextrin and cellulose.

Embodiments of the disclosure further include silage comprising an Acetobacter spp. inhibiting amount of Lactobacillus brevis (Levilactobacillus brevis) strain LB7148, deposited as Patent Deposit No. NRRL B-67991 and/or Lactobacillus buchneri (Lentilactobacillus buchneri) strain LN7149, deposited as Patent Deposit No. NRRL B-67992.

The present disclosure provides methods of treating silage for animal feed with the silage inoculant of the present disclosure, as well as the treated animal feed or silage itself. Often, the animal feed or silage will be whole plant corn silage (WPCS) or high moisture corn (HMC). The embodiments of the present disclosure also provide methods of improving animal performance by feeding the inoculated silage to an animal. Containers comprising the silage inoculant of the present disclosure and a carrier are also included.

Animals that are benefited by embodiments of the present disclosure are mammals and birds, including, but not limited to ruminant, equine, bovine, porcine, caprine, ovine and avian species, e.g., poultry.

The compositions which are used in the embodiments of the present disclosure may be in either liquid or dry form and may comprise additional bacterial strains. In solid treatment forms, the composition may comprise a mixed bacterial culture comprising Lactobacillus brevis (Levilactobacillus brevis) strain LB7148, deposited as Patent Deposit No. NRRL B-67991 and/or Lactobacillus buchneri (Lentilactobacillus buchneri) strain LN7149, deposited as Patent Deposit No. NRRL B-67992, together with a carrier.

The carrier may be in the nature of an aqueous or nonaqueous liquid or a solid. In solid forms, the composition may comprise solid carriers, solid diluents, or physical extenders. Examples of such solid carriers, solid diluents, or physical extenders include maltodextrin, starches, calcium carbonate, cellulose, whey, ground corn cobs, and silicone dioxide. Liquid carriers may be solutions, without limitation, in the form of emulsifiable concentrates, suspensions, emulsion including microemulsions, and/or suspoemulsions, and the like which optionally can be thickened into gels. In short, the carrier may be organic or an inorganic physical extender. The solid composition can be applied directly to the forage in the form of a light powder dusting, or if it is disbursed in a liquid carrier, it can successfully be sprayed on the forage.

Those of ordinary skill in the art will know of other suitable carriers and dosage forms, or will be able to ascertain such, using routine experimentation. Further, the administration of the various compositions can be carried out using standard techniques common to those of ordinary skill in the art.

An embodiment of the present disclosure is a composition for use as a silage inoculant comprising Lactobacillus brevis (Levilactobacillus brevis) strain LB7148, deposited as Patent Deposit No. NRRL B-67991 and/or Lactobacillus buchneri (Lentilactobacillus buchneri) strain LN7149, deposited as Patent Deposit No. NRRL B-67992, and/or a functional mutant thereof and a suitable carrier. In an embodiment of the present disclosure, the composition contains from about 10¹ to about 10¹⁰ viable organisms of the bacterial strain or functional mutant thereof per gram of a pre-ensiled plant material. In a further embodiment of the present disclosure, the composition contains from about 10² to about 10⁷ viable organisms of the bacterial strain or functional mutant thereof per gram of a pre-ensiled plant material. In yet a further embodiment of the present disclosure, the composition contains from about 10³ to about 10⁶ viable organisms of the bacterial strain or functional mutant thereof per gram of a pre-ensiled plant material.

Materials that are suitable for ensiling or storage, according to the methods of the present disclosure, are any which are susceptible to aerobic spoilage. The material will usually contain at least 25% by weight dry matter. Such materials include, but are not limited to, rye or traditional grass, maize, including high moisture corn, whole plant corn, alfalfa, wheat, legumes, cereals, oil seeds, sorghum, sunflower, barley, or other whole crop cereals. The silage storage management includes, but is not limited to, in bales (a form particularly susceptible to aerobic spoilage), oxygen limiting bags, bunkers, upright stave silos, oxygen limiting silos, bags, piles or any other form of storage which may be susceptible to aerobic spoilage.

The Acetobacter spp. inhibiting activity associated with the present disclosure may be found in other strains of Lactobacillus buchneri (Lentilactobacillus buchneri) and Lactobacillus brevis (Levilactobacillus brevis), in other species of Lactobacillus, e.g. Lactobacillus kefir, Lactobacillus parakefir, Lactobacillus parabuchneri, Lactobacillus sakei (Latilactobacillus sakei), Lactobacillus curvatus (Latilactobacillus curvatus), other species of lactic acid bacteria and possibly also in other genera.

As used herein, the term “strain” or “strain(s)” shall be interpreted to include any mutant or derivative of the bacterial strains disclosed herein, for example, Lactobacillus brevis (Levilactobacillus brevis) strain LB7148, Patent Deposit No. NRRL B-67991 and Lactobacillus buchneri strain LN7149. Patent Deposit No. NRRL B-67992 which retains the ability to inhibit the growth of Acetobacter spp. and the functional activity of improving aerobic stability of forage as described and defined by the methods and examples disclosed herein.

Lactobacillus brevis (Levilactobacillus brevis) strain LB7148, Patent Deposit No. NRRL B-67991 and Lactobacillus buchneri strain LN7149. Patent Deposit No. NRRL B-67992 of the embodiments of the present disclosure were each purified and isolated from corn.

After purification and isolation of the specific strains, taxonomic studies were done to identify the strains. Strains were identified as L. buchneri or L. brevis and given the prototype number LN7149 or LB7148, respectively. According to the compositions and methods of the present disclosure, these strain(s), compositions comprising these strain(s), and/or the factors produced by these strain(s), are used to treat forage materials.

### Deposits

Lactobacillus brevis (Levilactobacillus brevis) strain LB7148 and Lactobacillus buchneri (Lentilactobacillus buchneri) strain LN7149 were each deposited and were each accepted under the Budapest Treaty provisions on Oct. 29, 2020 with the Agricultural Research Service (ARS) Culture Collection (NRRL), housed in the National Center for Agricultural Utilization Research (NCAUR). Lactobacillus brevis (Levilactobacillus brevis) strain LB7148 was given Patent Deposit No. NRRL B-67991 and Lactobacillus buchneri (Lentilactobacillus buchneri) strain LN7149 was given Patent Deposit No. NRRL B-67992. The deposit collection address of NCAUR is 1815 N. University Street, Peoria, IL, 61604. The deposit(s) will irrevocably and without restriction or condition be available to the public upon issuance of a patent. However, it should be understood that the availability of a deposit does not constitute a license to practice the subject disclosure in derogation of patent rights granted by government action. Applicant(s) will meet all the requirements of 37 C.F.R. §§ 1.801-1.809, including providing an indication of the viability of the sample when the deposit(s) is made. Each deposit will be maintained without restriction in the NRRL Depository, which is a public depository, for a period of 30 years, or 5 years after the most recent request, or for the enforceable life of the patent, whichever is longer, and will be replaced if it ever becomes nonviable during that period.

Previously disclosed strain deposits include strain LN4637 given deposit number PTA-2494, as disclosed in U.S. Pat. No. 6,403,084; strain LN7125 given deposit number NRRL B-50733, as disclosed in U.S. Pat. No. 9,822,334 B2, strain LP286 given deposit number DSM18112, as disclosed in U.S. Pat. No. 5,747,020; and strain LP329 given deposit number ATCC55942, as disclosed in U.S. Pat. No. 5,747,020.

All publications and published patent documents mentioned in the specification are indicative of the level of those skilled in the art to which this disclosure pertains. All patents, publications, and published patent applications are herein incorporated by reference in the entirety to the same extent as if each individual patent, publication, or published patent application was specifically and individually indicated to be incorporated by reference in its entirety.

Although the foregoing disclosure has been described in some detail by way of illustration and example for purposes of clarity of understanding, certain changes and modifications may be practiced within the scope of the appended claims. The following examples are offered by way of illustration and not by way of limitation.

### EXAMPLES

The aspects of the disclosure are further defined in the following Examples, in which parts and percentages are by weight and degrees are Celsius, unless otherwise stated. These Examples, while indicating aspects of the disclosure, are given by way of illustration only. From the above discussion and these Examples, one skilled in the art can ascertain the essential characteristics of the aspects of the disclosure, and without departing from the spirit and scope thereof, can make various changes and modifications of them to adapt to various usages and conditions. Thus, various modifications in addition to those shown and described herein will be apparent to those skilled in the art from the foregoing description. Such modifications are also intended to fall within the scope of the appended claims.

### Example 1: Strain Selection

Lactic acid bacteria from the confidential and proprietary Forage Additive Research culture collection of Pioneer Hi-Bred International, Inc. were screened for inhibition of Acetobacter spp. isolates.

### Example 2: Effects of Lactic Acid Bacteria Silage Inoculants on Acetobacter Spp. Counts in Dried Ground Rehydrated Whole Plant Corn Silage Packets

Field harvested whole plant corn forage was dried at 60° C. for 48 hours and ground to 6 mm chop length. Dried ground whole plant corn forage consisting of a mixture of hybrids was rehydrated with sterile deionized water to a dry matter of 44% and stored at −80° C. for 1 month. Forage was thawed before use and starting pH was 4.82. Lactobacilus brevis (Levilactobacillus brevis) (LB), Lactococcus lactis (LL), Lactobacillus buchneri (Lentilactobacillus buchneri) (LN) strains were supplied as in-house fresh grown cultures. Each strain was applied to forage as a solution in Maximum Recovery Diluent (HiMedia Laboratories, Mumbai, India) to deliver an estimated 1×10⁵ CFU/g forage (log 5.00) when applied at a rate of 20 μL/g. The application dose, reported in log cfu/g silage, of bacterial inoculants on whole plant corn silage is provided in Table 1.

TABLE 1

| Treatment | Dose |
|---|---|
| Control | — |
| LB7148 | 5.05 |
| LL3116 | 5.73 |
| LL4539 | 5.90 |
| LN4867 | 3.94 |
| LN5549 | 4.92 |
| LN7149 | 4.16 |

An Acetobacter pasteurianus challenge was used in all packets, including control, and was applied at 6.10×10⁵ cfu/g forage (log 5.79) when applied at a rate of 20 μL/g. Each packet was filled with 10 gm of forage. All treatments were applied to forage directly in the packet and thoroughly mixed by finger squeezing for 15 seconds before sealing with a vacuum sealer (MiniPak®-Torre vacuum sealer, Dupey Equipment Co, Clive, IA). For each treatment and time point, two experimental 15×20.5 cm packets were filled, sealed, and stored at room temperature.

After 7, 14, 28, 63 and 84 days of ensiling, 20 ml of sterile Dnase/Rnase free water was added to each packet and the silage was thoroughly mixed by finger squeezing for 30 seconds. Silage plant particles were removed from the extract by pouring into a stomacher bag with filter lining (Seward, Bohemia, NY cat #BA6141/STR) and then decanting the extract. pH readings were taken on individual treatment reps using a benchtop pH meter (sympHony™ pH meter, VWR, Radnor, PA) and the average pH was reported. Acetobacter pasteurianus counts were obtained from treatment composites. Acetobacter pasteurianus counts were assessed on modified MRS agar (Hill and Hill, 1986), with aniline blue dye replacing cotton blue dye, after 6 days of aerobic incubation at 30° C. Acetobacter pasteurianus counts were expressed as log colony forming units per gram of forage.

Table 2 shows the effect of the bacterial inoculants on the pH of whole plant corn silage after 7, 14, 28, 63, and 84 days of ensiling. Metabolism of the substrate was demonstrated. The pH in untreated control dropped from day 7 to day 63 with a slight rise at day 84. LB7148 demonstrated the best pH decline, while LL3116 and LL4539 demonstrated the worst pH decline.

TABLE 2

| | pH | pH | pH | pH | pH |
|---|---|---|---|---|---|
| Treatment | Day 7 | Day 14 | Day 28 | Day 63 | Day 84 |
| Control | 4.98 | 4.77 | 4.45 | 4.36 | 4.43 |
| LB7148 | 3.99 | 4.04 | 3.96 | 4.01 | 4.08 |
| LL3116 | 5.06 | 4.83 | 4.65 | 4.55 | 4.45 |
| LL4539 | 5.03 | 4.89 | 4.58 | 4.53 | 4.45 |
| LN4867 | 4.65 | 4.34 | 4.52 | 4.56 | 4.55 |
| LN5549 | 4.46 | 4.40 | 4.51 | 4.39 | 4.49 |
| LN7149 | 4.53 | 4.44 | 4.38 | 4.30 | 4.39 |

Table 3 shows the effect of the inoculants on Acetobacter pasteurianus counts of whole plant corn silage, reported in log cfu/g silage, after 7, 14, 28, 63, and 84 days of ensiling. LB7148 reduced Acetobacter pasteurianus counts relative to control at all opening days except day 63, showing a 3.26 log difference at day 28 and an at least 3.10 log difference at day 84. LN7149 reduced Acetobacter pasteurianus counts relative control starting at day 14, with a 1.29 log difference at day 28 and a 2.93 log difference at day 84.LL3116, LL4539, LN4867 and LN5549 increased Acetobacter pasteurianus counts relative to control, with the exception of LN4867 at day 14 and day 28.

TABLE 3

| Treatment | Day 7 | Day 14 | Day 28 | Day 63 | Day 84 |
|---|---|---|---|---|---|
| Control | 5.52 | 5.99 | 5.64 | 3.61 | 4.58 |
| LB7148 | 5.44 | 5.20 | 2.38 | 4.38 | <1.48 |
| LL3116 | 6.99 | 6.54 | 6.62 | 6.41 | 5.94 |
| LL4539 | 7.10 | 7.16 | 6.53 | 6.42 | 5.98 |
| LN4867 | 6.43 | 5.58 | 4.95 | 6.55 | 5.57 |
| LN5549 | 7.41 | 6.32 | 6.31 | 6.13 | 6.12 |
| LN7149 | 5.66 | 5.56 | 4.35 | 2.57 | 1.65 |

### Example 3: Dna Profiling to Differentiate Bacteria at the Strain Level

Total DNA restriction enzyme digestion and electrophoresis was used to generate restriction fragment length total DNA profiles to differentiate Lactobacillus brevis (Levilactobacillus brevis) NRRL B-67991 (LB7148), Lactobacillus buchneri (Lentilactobacillus buchneri) NRRL B-67992 (LN7149), Lactobacillus buchneri (Lentilactobacillus buchneri) ATCC 202118 (LN3957), Lactobacillus brevis (Levilactobacillus brevis) NRRL B-30865 (LB1154), Lactobacillus buchneri (Lentilactobacillus buchneri) NRRL B-30866 (LN4888), Lactobacillus brevis (Levilactobacillus brevis) NRRL B-50731 (LB5328), Lactobacillus brevis (Levilactobacillus brevis) NRRL B-50732 (LB7123), Lactobacillus buchneri (Lentilactobacillus buchneri) NRRL B-50733 (LN7125), Lactobacillus buchneri (Lentilactobacillus buchneri) PTA-2493 (LN1391), Lactobacillus buchneri (Lentilactobacillus buchneri) PTA-2494 (LN4637), Lactobacillus buchneri (Lentilactobacillus buchneri) PTA-2495 (LN4750), and Lactobacillus buchneri (Lentilactobacillus buchneri) PTA-6138 (LN4017) bacterial strains.

1.5 ml of an overnight culture in MRS broth for each of Lactobacillus brevis (Levilactobacillus brevis) NRRL B-67991 (LB7148), Lactobacillus buchneri (Lentilactobacillus buchneri) NRRL B-67992 (LN7149), Lactobacillus buchneri (Lentilactobacillus buchneri) ATCC 202118 (LN3957), Lactobacillus brevis (Levilactobacillus brevis) NRRL B-30865 (LB1154), Lactobacillus buchneri (Lentilactobacillus buchneri) NRRL B-30866 (LN4888), Lactobacillus brevis (Levilactobacillus brevis) NRRL B-50731 (LB5328), Lactobacillus brevis (Levilactobacillus brevis) NRRL B-50732 (LB7123), Lactobacillus buchneri (Lentilactobacillus buchneri) NRRL B-50733 (LN7125), Lactobacillus buchneri (Lentilactobacillus buchneri) PTA-2493 (LN1391), Lactobacillus buchneri (Lentilactobacillus buchneri) PTA-2494 (LN4637), Lactobacillus buchneri (Lentilactobacillus buchneri) PTA-2495 (LN4750), and Lactobacillus buchneri (Lentilactobacillus buchneri) PTA-6138 (LN4017) was pelleted, washed with TE (10 mM Tris, pH 8.0, 1 mM EDTA, pH 8.0), and resuspended in 400 μl of TE. Then 5 μl 0.5 MEDTA, 12.5 μl 20% Triton X-100, and 20 μl mutanolysin (10 unit/μl in H2O) (Sigma) were added in sequence with vortexing after each addition and the tube was incubated at 37° C. for 2 hrs. Then 30 μl pronase (10 mg/ml) was added and incubated an additional 30 min at 37° C. 30 μl 20% w/v SDS, 50 mM Tris, pH 8.0, 20 mM EDTA, pH 8.0, was added and the tube was inverted several times and incubated at 65° C. for 10 min and then at 37° C. for 20 min. The mixture was extracted with 500 μl Tris-buffered phenol, pH 7.9 (Amresco) using Phase Lock Gel Light (Thermo Fisher NC1092951) and then with 500 μl chloroform/isoamyl alcohol (24:1) using Phase Lock Gel Heavy (Thermo Fisher NC1093153) for separation of the layers. The DNA was precipitated with EtOH, washed, and then resuspended in 50 μl H₂O containing 40 μg/ml RNase. The DNA was stored at 4° C.

DNA was quantitated with the Qubit dsDNA HS Assay Kit (Thermo Fisher) with 0.5 mL thin-wall tubes. The restriction digest was done with 700 ng or 17 μl DNA and 40 units Eco RI (Roche or Thermo Fisher) in the provided buffer and incubated for 2 hrs at 37° C.

A horizontal submarine gel for a large OnePhorAll gel box (Jordan Scientific Co.) was prepared with a 0.7% agarose gel (Seakem LE agarose, BMA) in 1×TAE (40 mM Tris-acetate, 2 mM EDTA) in a 20 cm (w)×30 cm (L) gel tray with a 0.8 mm tooth comb. Size standards (mixture of bacteriophage λ DNA and DNA Molecular Weight Marker IV (Sigma-Aldrich) were loaded on the left and right sides and in the center of the gel. After loading the Eco RI digests of Lactobacillus brevis (Levilactobacillus brevis) NRRL B-67991 (LB7148), Lactobacillus buchneri (Lentilactobacillus buchneri) NRRL B-67992 (LN7149), Lactobacillus buchneri (Lentilactobacillus buchneri) ATCC 202118 (LN3957), Lactobacillus brevis (Levilactobacillus brevis) NRRL B-30865 (LB1154), Lactobacillus buchneri (Lentilactobacillus buchneri) NRRL B-30866 (LN4888), Lactobacillus brevis (Levilactobacillus brevis) NRRL B-50731 (LB5328), Lactobacillus brevis (Levilactobacillus brevis) NRRL B-50732 (LB7123), Lactobacillus buchneri (Lentilactobacillus buchneri) NRRL B-50733 (LN7125), Lactobacillus buchneri (Lentilactobacillus buchneri) PTA-2493 (LN1391), Lactobacillus buchneri (Lentilactobacillus buchneri) PTA-2494 (LN4637), Lactobacillus buchneri (Lentilactobacillus buchneri) PTA-2495 (LN4750), and Lactobacillus buchneri (Lentilactobacillus buchneri) PTA-6138 (LN4017), the gel was run 16 hours at 60 volts in 1×TAE at room temperature with stirring with built-in stir bar, stained with ethidium bromide and imaged on a gel imager (Bio-Rad Gel Doc™ XR+).

Gel images were input into BioNumerics (version 7.5; Applied Maths). The reference standards for normalization were linear λ DNA and Roche Molecular Weight Marker IV. Due to the complexity of the Eco RI digest patterns, the Pearson product-moment correlation was used to calculate the similarity of DNA patterns. This method accounts for the whole shape of the gel lane profile rather than just the positions of each band. The unweighted pair group method using arithmetic averages (UPGMA) was used to construct dendrograms from the similarity matrix.

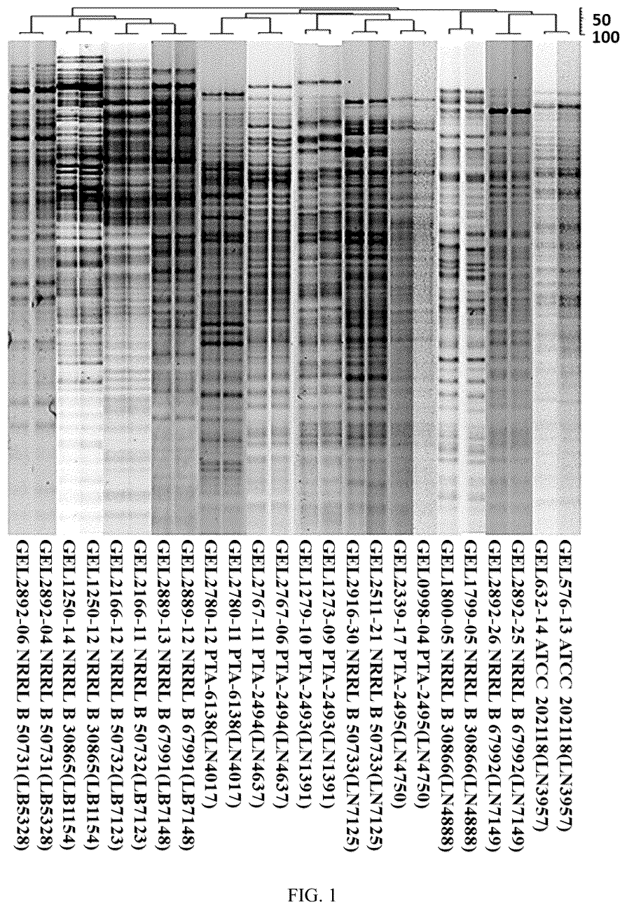

As shown in FIG. 1, Lactobacillus brevis (Levilactobacillus brevis) NRRL B-67991 (LB7148) and Lactobacillus buchneri (Lentilactobacillus buchneri) NRRL B-67992 (LN7149) each have a unique Eco RI total DNA profile, which differs from each of the profiles generated by Lactobacillus buchneri (Lentilactobacillus buchneri) ATCC 202118 (LN3957), Lactobacillus brevis (Levilactobacillus brevis) NRRL B-30865 (LB1154), Lactobacillus buchneri (Lentilactobacillus buchneri) NRRL B-30866 (LN4888), Lactobacillus brevis (Levilactobacillus brevis) NRRL B-50731 (LB5328), Lactobacillus brevis (Levilactobacillus brevis) NRRL B-50732 (LB7123), Lactobacillus buchneri (Lentilactobacillus buchneri) NRRL B-50733 (LN7125), Lactobacillus buchneri (Lentilactobacillus buchneri) PTA-2493 (LN1391), Lactobacillus buchneri (Lentilactobacillus buchneri) PTA-2494 (LN4637), Lactobacillus buchneri (Lentilactobacillus buchneri) PTA-2495 (LN4750), and Lactobacillus buchneri (Lentilactobacillus buchneri) PTA-6138 (LN4017). Each of these microbial strains were determined to be unique and different strains by Eco RI total DNA profiling (see, Chan, R. K., Wortman, C. R., Smiley B. K., & Hendrick, C. A. 2003. Construction and use of a computerized DNA fingerprint database for lactic acid bacteria from silage. J. of Microbiol. Methods 55:565-574).

### Example 4: Effects of Lactobacillus Microbial Strains on Fermentation and Aerobic Stability of Whole Plant Corn Silage

Based on the results, testing to study the impact of Lactobacillus microbial strains on fermentation and aerobic stability of whole plant corn silage (WPCS) was done in mini-silos using a high dry matter, low packing density, aerobically and microbially challenged silage model. The trial was performed at the experimental farm of the University of Turin in northern Italy. Corn was directly harvested as chopped whole crop with a precision forage harvester at 12 mm chopping length. The DM content of the forage at harvest was 41.1% and pH was 6.02. Yeast, mold and lactic acid bacteria counts were higher than 7 log cfu/g, whereas acetic acid bacteria and enterobacteria were higher than 8 log cfu/g at harvest.

Lactic acid bacteria treatments were grown and freeze dried according to standard methods and were resuspended in water before use. A mixture of four acetic acid bacteria (AAB) Acetobacter pasteurianus challenge strains were fresh grown. All treatments were applied by spraying uniformly onto pre-ensiled plant material (whole plant corn forage) with a hand sprayer at doses shown in Table 4. All silages, including the control silage, were treated with an AAB challenge at an approximate dose of 1.0×10⁷ cfu/g forage. The forage was constantly hand-mixed during inoculation.

TABLE 4

| TRT | Strain Name (s) | Dose (cfu/g forage) |
|---|---|---|
| 1 | None (Control) | None |
| 2 | LN4637 + LN7125 + LP286 + LP329 | 1.1 × 10⁵ |
| | (Positive Control) | |
| 3 | LB7148 | 1.0 × 10⁵ |
| 4 | LN7149 | 1.0 × 10⁵ |
| 5 | LB7148 + LP286 + LP329 | 1.1 × 10⁵ |
| 6 | LN7149 + LP286 + LP329 | 1.1 × 10⁵ |
| 7 | LB7148 + LN7125 + LP286 + LP329 | 1.1 × 10⁵ |
| 8 | LN7149 + LN7125 + LP286 + LP329 | 1.1 × 10⁵ |

Forage was hand-packed at an approximate packing density of 400 kg FM/m³ (144 kg DM/m³) in 20-liter plastic silos equipped with holes for air infusion and a lid that enabled gas release. Three replicates were ensiled per treatment. Silos were stored at ambient temperature (20±1° C.) for 118 days. An air infusion (AI) challenge to facilitate spoilage was conducted at days 28 and 42. The air infusion was made by two holes of 8 mm diameter in the side of the silos. Air infusion occurred by removing tape (ag bag silo repair tape) covering each of the 2 holes at opposite ends of the silo, for a 24-hour period.

After 118 days of ensiling, silos were emptied, silage was thoroughly mixed and sub-sampled to determine the DM content, fermentation profile, microbial counts, and the aerobic stability. A 30 g sample was transferred into a sterile homogenization bag, suspended 1:9 w/v in a peptone salt solution (1 g of bacteriological peptone and 9 g of sodium chloride per liter) and homogenized for 4 min in a laboratory Stomacher blender (Seward Ltd, London, UK). Serial dilutions were prepared. The mold and yeast numbers were determined using the pour plate technique with 40.0 g/L of Yeast Extract Glucose Chloramphenicol Agar (YGC agar, DIFCO, West Molesey, Surrey, UK) after incubation at 25° C. for 3 and 5 days for yeast and mold, respectively. Yeast and mold colony forming units (cfu) were enumerated separately, according to their macromorphological features, on plates. The lactic acid bacteria (LAB) were determined on MRS agar (Merck, Whitehouse Station, NY) with added natamycin (0.25 g/L), by incubating Petri plates at 30° C. for 3 days under anaerobic conditions, according to Spoelstra et al. (1988). Enterobacteria count was determined on Violet Red Bile Dextrose agar (Merck, Whitehouse Station, NY) and acetic acid bacteria (AAB) were counted following Spoelstra et al. (1988) with minor modifications.

Aerobic stability was determined by monitoring the temperature increase due to the microbial activity of the samples exposed to air. About two to three kilograms of each silo was allowed to aerobically deteriorate in a controlled temperature room (20° C.±1° C.) in 17-liter polystyrene boxes for 7 days. A single layer of aluminum cooking foil was placed over each box to prevent drying and dust contamination, but also to allow air penetration. The temperature of the room and of the silage was measured each hour by a data logger. Aerobic stability was defined as the number of hours the silage remained stable before rising more than 2° C. above room temperature (Ranjit and Kung, 2000). A higher time is desirable indicating longer time before spoilage occurs. The integration of the area between the actual silage temperature curve and the line drawn by ambient temperature was calculated (cummDD). A lower cummDD is desirable indicating less total heating.

After 118 d of conservation, good fermentation was demonstrated with pH below 4.0 in all treatments, including the control. Chemical and fermentative characteristics of corn silage after 118 days of conservation are provided in Table 5 (different lower-case letters indicate results are significantly different). A statistically lower lactic-to-acetic ratio and increased acetic acid level were detected in TRT 2, with numerical improvements in TRT 4, 5, 6, 8. Acetic acid levels improved in TRT 2, 4, 5, 6, and 8. The chemical 1,2-propanediol was found in TRT 2, and TRT 8.

TABLE 5

| | | | | | | 1,2- | |
|---|---|---|---|---|---|---|---|
| | DM | | Lactic acid | Acetic acid | Lactic/acetic | propanediol | Ethanol |
| | (%) | pH | (g/kg DM) | (g/kg DM) | ratio | (g/kg DM) | (g/kg DM) |
| TRT 1 | 39.8 | 3.82 | 36.1^{ab} | 8.5^{c} | 4.22^{ab} | <0.1 | 6.6 |
| TRT 2 | 39.8 | 3.85 | 31.2^{b} | 14.2^{ab} | 2.28^{c} | 0.9 | 4.9 |
| TRT 3 | 40.6 | 3.81 | 34.9^{ab} | 8.0^{c} | 4.39^{a} | <0.1 | 7.0 |
| TRT 4 | 40.7 | 3.84 | 38.2^{ab} | 9.1^{bc} | 4.18^{ab} | <0.1 | 7.1 |
| TRT 5 | 40.0 | 3.84 | 39.1^{ab} | 10.6^{bc} | 3.79^{ab} | <0.1 | 6.7 |
| TRT 6 | 40.1 | 3.81 | 40.8^{ab} | 10.6^{bc} | 3.88^{ab} | <0.1 | 5.4 |
| TRT 7 | 39.8 | 3.81 | 34.3^{ab} | 8.4^{c} | 4.11^{ab} | <0.1 | 7.6 |
| TRT 8 | 40.2 | 3.81 | 35.4^{ab} | 10.7^{bc} | 3.56^{ab} | 0.3 | 6.1 |
| P-value | 0.155 | 0.856 | 0.022 | 0.004 | <0.001 | — | 0.056 |

Microbial characteristics and aerobic stability of corn silage after 118 days of conservation are provided in Table 6. Yeast count was on average 5.2 log cfu/g with the greatest reductions relative to control found in TRT 6 and TRT 8. Numerical reductions were observed in acetic acid bacteria levels for all treatments relative to control. Control silage showed 40 hours of aerobic stability. Lactobacillus strain combinations numerically improved silage aerobic stability, with the greatest improvement relative to control (TRT 1) in TRT 2 (+29 h), TRT 6 (+15 h) and TRT 8 (+25 h). These treatments, along with TRT 7,also showed the greatest reduction in total heating time relative to the control.

TABLE 6

| | | | Acetic acid | Entero- | Lactic acid | Aerobic | Heating |
|---|---|---|---|---|---|---|---|
| | Yeast | Mold | bacteria | bacteria | bacteria | stability | Time |
| | (log cfu/g) | (log cfu/g) | (log cfu/g) | (log cfu/g) | (log cfu/g) | (h) | (cummDD) |
| TRT 1 | 5.75 | 2.05 | 5.65 | 0.67 | 7.54^{abc} | 40 | 1235 |
| TRT 2 | 5.13 | 1.66 | 4.90 | 1.42 | 8.76^{ab} | 69 | 721 |
| TRT 3 | 5.06 | <1.00 | 4.82 | 1.03 | 7.45^{abc} | 44 | 1047 |
| TRT 4 | 5.36 | 1.00 | 4.72 | 0.50 | 8.13^{abc} | 40 | 1169 |
| TRT 5 | 5.78 | 2.46 | 5.56 | 0.50 | 7.86^{abc} | 46 | 1141 |
| TRT 6 | 4.41 | 1.33 | 5.16 | 0.50 | 9.15^{a} | 55 | 929 |
| TRT 7 | 5.33 | 1.00 | 5.32 | 0.50 | 9.12^{a} | 46 | 913 |
| TRT 8 | 3.96 | <1.00 | 4.90 | 0.50 | 8.49^{abc} | 65 | 710 |
| P-value | 0.234 | — | 0.423 | — | <0.001 | 0.068 | 0.096 |

The foregoing demonstrates the impact of Lactobacillus microbial strains on aerobic stability using a silage spoilage model in which high dry matter, low packing density, air infusion and high acetic acid bacteria provided a spoilage challenge. Acetic acid bacteria were naturally present in silages at a level higher than 8 log cfu/g, and an additional laboratory grown dose was included to ensure sufficient levels. Despite these difficult working conditions, the treatments evaluated showed numerical differences from control in aerobic stability, yeast counts and acetic acid bacteria counts after 118 d of conservation, with TRT 2, 6, 7 and 8 providing the strongest protection against spoilage and silage heating.

Having illustrated and described the principles of the embodiments of the present disclosure, it should be apparent to persons skilled in the art that the embodiments of the disclosure can be modified in arrangement and detail without departing from such principles. Thus, the present disclosure encompasses all alternate embodiments that fall literally or equivalently within the scope of these claims.

It is understood that various preferred embodiments are shown and described above to illustrate different possible features of the present disclosure and the varying ways in which these features may be combined. Apart from combining the different features of the above embodiments in varying ways, other modifications are also considered to be within the scope of the present disclosure.

1. A composition comprising: a first bacterial strain, a pre-ensiled plant material, and a suitable carrier, wherein the first bacterial strain is any one of or a combination of Lactobacillus brevis (Levilactobacillus brevis) strain LB7148, deposited as Patent Deposit No. NRRL B-67991 and Lactobacillus buchneri (Lentilactobacillus buchneri) strain LN7149, deposited as Patent Deposit No. NRRL B-67992.

2. The composition of claim 1, further comprising a second bacterial strain, wherein the second bacterial strain is selected from one or more of a Lactobacillus buchneri (Lentilactobacillus buchneri) strain, a Lactobacillus plantarum (Lactiplantibacillus plantarum) strain, a Lactobacillus alimentarius strain, a Lactobacillus crispatus strain, a Lactobacillus paralimentarius strain, a Lactobacillus brevis (Levilactobacillus brevis) strain, or an Enterococcus facium strain.

3. The composition of claim 1, further comprising a yeast strain, wherein the yeast strain is selected from one or more of Saccharomyces cerevisiae strain YE206, deposited as Patent Deposit No. NRRL Y-50734; Saccharomyces cerevisiae strain YE 1241, deposited as Patent Deposit No. NRRL Y-50735; or Saccharomyces cerevisiae strain YE1496, deposited as Patent Deposit No. NRRL Y-50736.

4. The composition of claim 1, wherein composition comprises a second bacterial strain and the second bacterial strain further comprises one or more of Lactobacillus plantarum (Lactiplantibacillus plantarum) strain LP287, Lactobacillus plantarum (Lactiplantibacillus plantarum) strain LP318, Lactobacillus plantarum(Lactiplantibacillus plantarum) strain LP319, Lactobacillus plantarum (Lactiplantibacillus plantarum) strain LP346, Lactobacillus plantarum (Lactiplantibacillus plantarum) strain LP347, Lactobacillus plantarum (Lactiplantibacillus plantarum) strain LP286, Lactobacillus buchneri (Lentilactobacillus buchneri) strain LN4017, Lactobacillus buchneri (Lentilactobacillus buchneri) strain LN4637, Lactobacillus plantarum (Lactiplantibacillus plantarum) strain LP329, Lactobacillus plantarum (Lactiplantibacillus plantarum) strain LP7109, Lactobacillus buchneri (Lentilactobacillus buchneri) strain LN7125, Lactobacillus brevis (Levilactobacillus brevis) strain LB7123, Lactobacillus brevis (Levilactobacillus brevis) strain LB5328, or Lactobacillus buchneri (Lentilactobacillus buchneri) strain LN5689.

5. The composition of claim 1, wherein the composition comprises from about 10¹ to about 10¹⁰ viable organisms of the first bacterial strain per gram of the pre-ensiled plant material.

6. The composition of claim 5, wherein the composition comprises from about 10³ to about 10⁶ viable organisms of the first bacterial strain per gram of the pre-ensiled plant material.

7. The composition of claim 1, wherein the pre-ensiled plant material is selected from the group consisting of grasses, maize, alfalfa, wheat, legumes, sorghum, sunflower, barley, grains, and any mixture thereof.

8. A method for treating a pre-ensiled plant material, the method comprising adding to the pre-ensiled plant material a first bacterial strain and a suitable carrier, wherein the first bacterial strain is any one of or a combination of Lactobacillus brevis (Levilactobacillus brevis) strain LB7148, deposited as Patent Deposit No. NRRL B-67991 and Lactobacillus buchneri (Lentilactobacillus buchneri) strain LN7149, deposited as Patent Deposit No. NRRL B-67992.

9. The method of claim 8, further comprising adding a second bacterial strain to the pre-ensiled plant material, wherein the second bacterial strain is selected from one or more of a Lactobacillus buchneri (Lentilactobacillus buchneri) strain, a Lactobacillus plantarum (Lactiplantibacillus plantarum) strain, a Lactobacillus alimentarius strain, a Lactobacillus crispatus strain, a Lactobacillus paralimentarius strain, a Lactobacillus brevis (Levilactobacillus brevis) strain, or an Enterococcus facium strain.

10. The method of claim 8, further comprising adding a yeast strain to the pre-ensiled plant material, wherein the yeast strain is selected from one or more of Saccharomyces cerevisiae strain YE206, deposited as Patent Deposit No. NRRL Y-50734; Saccharomyces cerevisiae strain YE 1241, deposited as Patent Deposit No. NRRL Y-50735; or Saccharomyces cerevisiae strain YE1496, deposited as Patent Deposit No. NRRL Y-50736.

11. The method of claim 9, wherein the second bacterial strain comprises one or more of Lactobacillus plantarum (Lactiplantibacillus plantarum) strain LP287, Lactobacillus plantarum (Lactiplantibacillus plantarum) strain LP318, Lactobacillus plantarum (Lactiplantibacillus plantarum) strain LP319, Lactobacillus plantarum (Lactiplantibacillus plantarum) strain LP346, Lactobacillus plantarum (Lactiplantibacillus plantarum) strain LP347, Lactobacillus plantarum (Lactiplantibacillus plantarum) strain LP286, Lactobacillus buchneri (Lentilactobacillus buchneri) strain LN4017, Lactobacillus buchneri (Lentilactobacillus buchneri) strain LN4637, Lactobacillus plantarum (Lactiplantibacillus plantarum) strain LP329, Lactobacillus plantarum (Lactiplantibacillus plantarum) strain LP7109, Lactobacillus buchneri (Lentilactobacillus buchneri) strain LN7125, Lactobacillus brevis (Levilactobacillus brevis) strain LB7123, Lactobacillus brevis (Levilactobacillus brevis) strain LB5328, or Lactobacillus buchneri (Lentilactobacillus buchneri) strain LN5689.

12. The method of claim 8, wherein the first bacterial strain comprises from about 10¹ to about 10¹⁰ viable organisms of the first bacterial strain per gram of the pre-ensiled plant material.

13. The method of claim 12, wherein the first bacterial strain comprises from about 10³ to about 10⁶ viable organisms of the first bacterial strain per gram of the pre-ensiled plant material.

14. The method of claim 8, wherein the pre-ensiled plant material is selected from the group consisting of grasses, maize, alfalfa, wheat, legumes, sorghum, sunflower, barley, grains, and mixtures thereof.

15. A method of improving meat and milk performance in an animal, the method comprising feeding the animal silage, wherein the silage comprises a pre-ensiled plant material treated with an inoculant comprising a first bacterial strain and a suitable carrier, wherein the first bacterial strain is any one of or a combination of Lactobacillus brevis (Levilactobacillus brevis) strain LB7148, deposited as Patent Deposit No. NRRL B-67991 and Lactobacillus buchneri (Lentilactobacillus buchneri) strain LN7149, deposited as Patent Deposit No. NRRL B-67992.

16. The method of claim 15, wherein the inoculant further comprises a second bacterial strain selected from one or more of a Lactobacillus buchneri (Lentilactobacillus buchneri) strain, a Lactobacillus plantarum (Lactiplantibacillus plantarum) strain, a Lactobacillus alimentarius strain, a Lactobacillus crispatus strain, a Lactobacillus paralimentarius strain, a Lactobacillus brevis (Levilactobacillus brevis) strain, or an Enterococcus facium strain, and mixtures thereof.

17. The method of claim 15, wherein the inoculant further comprises a yeast strain selected from one or more of Saccharomyces cerevisiae strain YE206, deposited as Patent Deposit No. NRRL Y-50734; Saccharomyces cerevisiae strain YE 1241, deposited as Patent Deposit No. NRRL Y-50735; or Saccharomyces cerevisiae strain YE1496, deposited as Patent Deposit No. NRRL Y-50736, and mixtures thereof.

18. The method of claim 15, wherein the inoculant further comprises a second bacterial strain comprising one or more of Lactobacillus plantarum (Lactiplantibacillus plantarum) strain LP287, Lactobacillus plantarum (Lactiplantibacillus plantarum) strain LP318, Lactobacillus plantarum (Lactiplantibacillus plantarum) strain LP319, Lactobacillus plantarum (Lactiplantibacillus plantarum) strain LP346, Lactobacillus plantarum (Lactiplantibacillus plantarum) strain LP347, Lactobacillus plantarum (Lactiplantibacillus plantarum) strain LP286, Lactobacillus buchneri (Lentilactobacillus buchneri) strain LN4017, Lactobacillus buchneri (Lentilactobacillus buchneri) strain LN4637, Lactobacillus plantarum (Lactiplantibacillus plantarum) strain LP329, Lactobacillus plantarum (Lactiplantibacillus plantarum) strain LP7109, Lactobacillus buchneri (Lentilactobacillus buchneri) strain LN7125, Lactobacillus brevis (Levilactobacillus brevis) strain LB7123, Lactobacillus brevis (Levilactobacillus brevis) strain LB5328, or Lactobacillus buchneri (Lentilactobacillus buchneri) strain LN5689.

19. The method of claim 15, wherein the inoculant contains from about 10¹ to about 10¹⁰ viable organisms of the first bacterial strain per gram of the pre-ensiled plant material or wherein the pre-ensiled plant material is selected from the group consisting of grasses, maize, alfalfa, wheat, legumes, sorghum, sunflower, barley, grains, and mixtures thereof.

20. The method of claim 19, wherein the inoculant contains from about 10³ to about 10⁶ viable organisms of the first bacterial strain per gram of the pre-ensiled plant material.

21. (canceled).
